(19) United States
(12) Patent Application Publication
(10) Pub. No.: US 2009/0212124 A1
(43) Pub. Date: Aug. 27, 2009

(54) APPARATUS AND METHOD FOR DISPENSING A VOLATILE COMPOSITION

(76) Inventor: Kevin Brian KENNY, Cleveland, MO (US)

Correspondence Address: SHELL OIL COMPANY, P O BOX 2463, HOUSTON, TX, 772522463, US

(21) Appl. No.: 12/208,178

(22) Filed: Sep. 10, 2008

Related U.S. Application Data
(60) Provisional application No. 60/971,765, filed on Sep. 12, 2007.

(51) Int. Cl. A61L 9/04

(52) U.S. Cl. 239/58

(57) ABSTRACT

An apparatus comprising: a base having a plurality of compartments; a volatile composition located in at least one of the plurality of compartments; and a first member in a substantially coaxial arrangement with the base, the first member having a first opening exposing at least a portion of at least one compartment to a surrounding atmosphere. A method comprising: placing a volatile composition in at least one of a plurality of compartments of a base; and rotating a first member in a substantially coaxial arrangement with the base such that a first opening in the first member exposes at least a portion of at least one compartment to a surrounding atmosphere.

### CROSS-REFERENCE TO RELATED APPLICATION

This application claims the benefit of U.S. Provisional Application No. 60/971,765, filed Sep. 12, 2007, which is incorporated herein by reference.

### FIELD OF THE INVENTION

The present invention relates to an apparatus and method for dispensing one or more volatile compositions.

### BACKGROUND OF THE INVENTION

A variety of dispensers are available for dispensing volatile compositions in a variety of contexts. One context is an air freshener dispenser to freshen the air in a home or a vehicle.

Some dispensers have minimal parts, operate passively, or use the flow of air from an air conditioner or vent. Some dispensers have mechanical and/or electrical parts and actively dispense the volatile composition.

Many dispensers can dispense only a single volatile composition. Where the volatile composition is a fragrance material, single fragrance dispensers suffer from the drawback that, after a relatively brief period of time, people become desensitized to a given fragrance. This phenomenon is called “fragrance fatigue.”

One approach to reducing fragrance fatigue has been to modify single fragrance dispensers to incorporate an interval during which no fragrance is emitted. Another approach has been to develop dispensers that emit multiple fragrances.

Unfortunately, dispensers that emit multiple fragrances tend to be relatively complex and/or to require a relatively large number of parts. As a result, the manufacture and/or operation of such dispensers tend to be relatively costly compared to single fragrance dispensers. See, for example, U.S. Pat. No. 6,950,607 wherein a multi-fragrance scent dispenser includes a housing having an outlet opening; first and second holders in the housing for holding first and second liquid fragrance containers having first and second wicks, respectively, extending out of the first and second liquid fragrance containers; a first heater positioned in the housing to be in surrounding relation to the first wick when the first liquid fragrance container is held by the first holder; a second heater positioned in the housing to be in surrounding relation to the second wick when the first liquid fragrance container is held by the first holder; a control circuit which selectively controls activation of the first and second heaters; and a blocking plate movable relative to the heaters for at least partially blocking escape of evaporated fragrance through the outlet opening from at least one of the first and second wicks.

### SUMMARY OF THE INVENTION

In some embodiments, the present invention provides an apparatus comprising: a base having a plurality of compartments; a volatile composition located in at least one of the plurality of compartments; and a first member in a substantially coaxial arrangement with the base, the first member having a first opening exposing at least a portion of at least one compartment to a surrounding atmosphere.

In other embodiments, the present invention provides an apparatus comprising: a base having a plurality of compartments wherein at least one of the plurality of compartments contains a volatile composition; a first member in a substantially coaxial arrangement with the base, the first member having a first opening exposing a portion of two contiguous compartments to a surrounding atmosphere; and a second member substantially coaxial with the first member having a second opening rotatably positionable relative to the first opening adjusting an overlap area.

In yet other embodiments, the present invention provides a method comprising: placing a volatile composition in at least one of a plurality of compartments of a base; and rotating a first member in a substantially coaxial arrangement with the base such that a first opening in the first member exposes at least a portion of at least one compartment to a surrounding atmosphere.

### BRIEF DESCRIPTION OF THE DRAWINGS

For a detailed understanding of embodiments of the present invention, reference is made to the following detailed description of example embodiments, taken in conjunction with the accompanying drawings, in which like elements have been given like numerals, wherein:

FIG. 1 is a schematic diagram of an example of a dispenser for dispensing one or more volatile compositions;

FIG. 2A is a view of a base of the dispenser of FIG. 1;

FIG. 2B is a cross section view of the base of FIG. 2A;

FIG. 3A is a view of the first member of the dispenser of FIG. 1;

FIG. 3B is a cross section view of the first member of FIG. 3A;

FIG. 4 is a view of a dispenser having a first member exposing a portion of the base;

FIG. 5 is a top view of the assembled dispenser of FIG. 1;

FIG. 6 is a schematic diagram of a side view of a dispenser comprising a clip to attach the dispenser to an air vent FIG. 7 is a schematic diagram of a first member having multiple openings;

FIG. 8A is a schematic diagram of a fragrance fin located in a compartment in a base;

FIG. 8B is a diagram depicting a light pipe effect of the fragrance fin of FIG. 8B;

FIG. 9 is a schematic diagram depicting another example of a dispenser for dispensing one or more volatile compositions;

FIG. 10A is a view of a base of the dispenser of FIG. 9;

FIG. 10B is a cross-section view of the base of FIG. 10A;

FIG. 11A is a view of the second member of FIG. 9;

FIG. 11B is a side view of the second member of FIG. 11A;

FIG. 12A is a view of the first member of FIG. 9;

FIG. 12B is a side view of the first member of FIG. 12A;

FIG. 13 is a partial cut-away view showing the relationship of the parts of FIG. 9;

FIG. 14 is a schematic diagram of another example of a dispenser for dispensing one or more volatile compositions; and

FIG. 15 is a view of an example showing the location of selected parts of a dispenser.

### DETAILED DESCRIPTION OF THE INVENTION

The present application discloses illustrative examples of dispensers for dispensing one or more volatile compositions. These examples are intended to be illustrative, and are not intended to limit the invention to the described embodiments. The dispensers may be used to dispense a wide variety of volatile compositions. In one embodiment, the volatile composition is slowly diffused into the surrounding atmosphere. Suitable volatile compositions include any composition with a relatively low molecular weight which can be volatilized. Suitable volatile compositions include, for example, fragrance materials, disinfectants, and pesticides. As used herein, the term fragrance material refers to the base material, which may be in liquid or solid form, and the term fragrance refers to the scent, or vapor, emitted therefrom.

A fragrance material can be relatively simple in composition. A fragrance material also can be a complex mixture of natural and/or synthetic chemical components. Suitable fragrance materials include, for example, synthetic or naturally-derived volatile essential oils. For example, an essential oil may be any concentrated liquid containing volatile aroma compounds from plants, and the liquid is commonly known as “oil of” the plant material from which the liquid is extracted. Examples include oil of bergamot, bitter orange, lemon, mandarin, caraway, cedar leaf, clove leaf, cedar wood, geranium, lavender, orange, origanum, petitgrain, white cedar, patchouli, lavandin, neroli, and rose absolute. Suitable natural botanical fragrance materials also can be employed, for example, those selected from the group consisting of eucalyptus, jasmine-lavender, wintergreen, spearmint, wormwood, echinacea, lemongrass, calendula, balsam, oleoresins, tea tree, ginseng, licorice, and menthol.

Suitable artificial liquid fragrance materials include, for example, geraniol, geranyl acetate, eugenol, isoeugenol, linalool, linalyl acetate, phenethyl alcohol, and methylionone. Any other fragrance material not specifically mentioned herein but having similar characteristics, configurations, and/or structures can be used with equal efficacy.

A liquid fragrance material can be formed into a thixotropic gel by the addition of a conventional thickening agent. Suitable thickening agents include, for example, a water base gel, a polymer base gel, a polyamide, scented polyvinyl chloride (PVC) beads, scented ethylene vinyl acetate (EVA) beads, and silica.

The fragrance material also can be in the form of a crystalline solid, which has the ability to sublime into the vapor phase at ambient temperatures. A crystalline fragrance material can be selected from organic compounds which include vanillin, ethyl vanillin, coumarin, tonalide, calone, musk xylol, musk ketone benzophenone, raspberry ketone, methyl naphthyl ketone beta, phenyl ethyl salicylate, veltol, maltol, and maple lactone.

Referring for purposes of illustration to an automotive air freshener, the dispenser may be adjusted to dispense one fragrance, two fragrances, or more than two fragrances into the surrounding atmosphere. In one embodiment the dispenser contains a plurality of fragrance materials and the user can adjust the dispenser to dispense one single fragrance or a combination of at least two fragrances emitted from the fragrance materials.

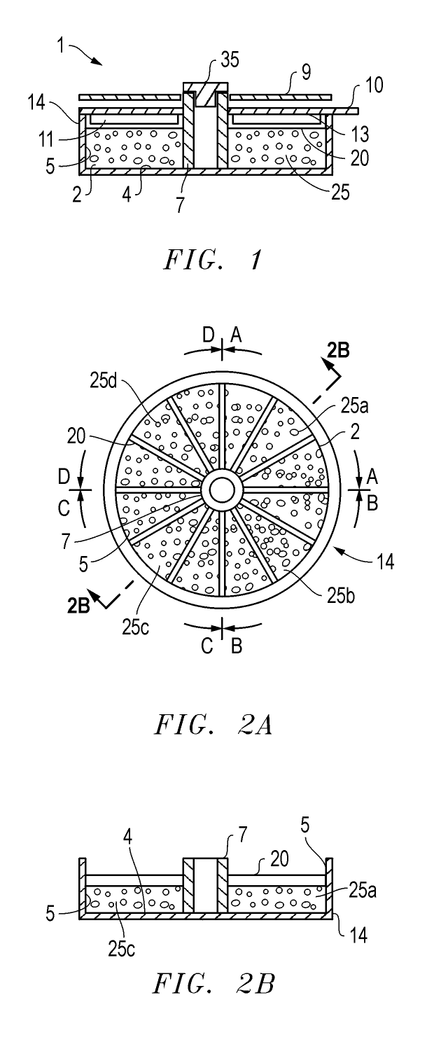

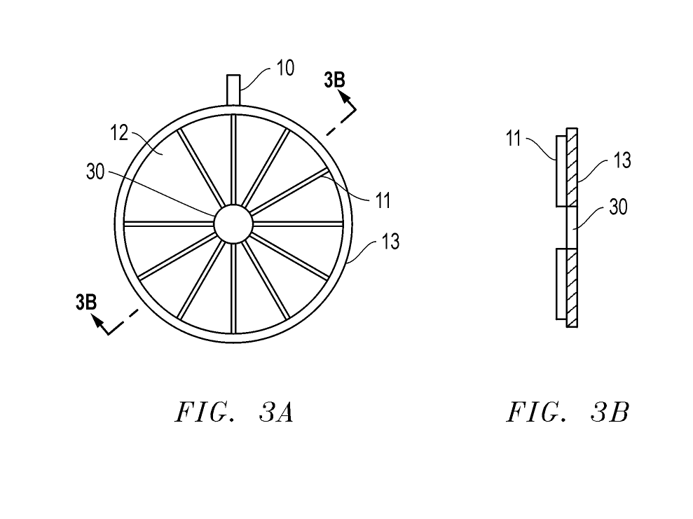

Referring to FIG. 1, a dispenser 1 comprises a base 14, having a plurality of dividers 20, also called dividing walls, separating the base 14 into multiple compartments 2, also called cavities. Any number of compartments 2 may be provided. It is understood that the specific embodiments described herein are for illustration, and are not to be construed as limiting the number of compartments in the present invention. In one illustrative example, each compartment 2 contains a volatile composition 25. Volatile composition 25 may be a fragrance material as described above. In one embodiment, a compartment 2 may contain a fragrance material different from that contained in at least one other compartment 2. Alternatively, any number of compartments may contain the same fragrance material. The dispenser also comprises a first member 13 having an opening (see FIG. 3A) that may expose more than one of the plurality of compartments 2. In one embodiment, first member 13 is substantially circular in shape.

Referring also to FIGS. 2A and 2B, base 14 has multiple pie shaped compartments 2. In one embodiment, each compartment 2 retains a single fragrance material. In one embodiment, compartments 2 radiate outward from the center post 7 and are filled with fragrance materials. For purposes of illustration, the base may contain, for example, twelve compartments 2, with four different volatile compositions such as fragrance materials 25a, 25b, 25c, 25d, each situated in three consecutive compartments indicated by the circumferential markers A, B, C, D, respectively. As one proceeds clockwise around, for example, twelve compartments 2 with dividers 20 in base 14, there may be three consecutive compartments with fragrance material 25a, three consecutive compartments with fragrance material 25b, three consecutive compartments with fragrance material 25c, and three consecutive compartments with fragrance material 25d.

In the embodiment of the invention represented in FIGS. 1-2B, base 14 has a bottom 4 and side 5. Center post 7 positions first member 13 (FIGS. 3A and 3B) on base 14 and permits first member 13 and base 14 to be rotatable relative to each other. Cap 35 axially retains first member 13 and second member 9 (discussed below), when included.

Referring also to FIGS. 3A and 3B, the bottom side of first member 13 has a pattern of ridges 11 extending radially outward from center hole 30 in a pattern similar to that of dividers 20. When first member 13 is assembled on center post 7, ridges 11 extend in close proximity to dividers 20 and act to form a barrier seal between the different compartments 2 in base 14, so that unselected fragrance materials are substantially prevented from emitting their fragrances to the surrounding atmosphere.

Alternatively, in another embodiment, ridges 11 may be sized to extend into contact with dividers 20 in the assembled condition. This embodiment provides positive indication to the user in making fragrance adjustments to the apparatus. For example, rotating base 14 will produce a clicking noise and vibration sensation informing a user that one adjustment has been successfully completed. As base 14 is rotated further, a click is emitted every time the ridges 11 of first member 13 pass a divider 20. In one example embodiment, tab 10 is used to facilitate rotational adjustment of first member 13.

First member 13 also comprises opening 12. When assembled to base 14, opening 12 is sized to expose one or more compartments 2 having a volatile composition 25 therein to ambient air. In one embodiment, opening 12 in first member 13 is equal to about one-quarter of the surface area of first member 13. Alternatively, opening 12 may be sized to expose any predetermined number of compartments 2.

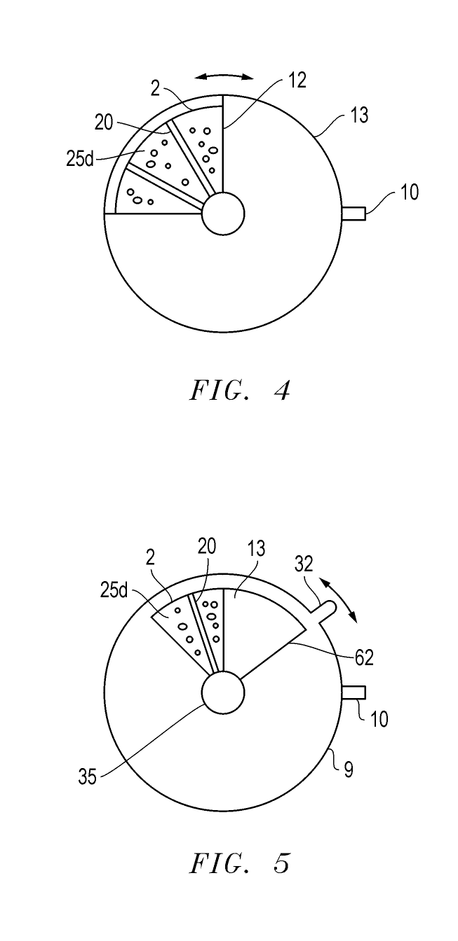

In one illustrative example, also referring to FIG. 4, first member 13 is shown in an assembled position with opening 12 adjusted to expose three compartments 2. Each exposed compartment 2 has fragrance material 25d therein. Other quadrants of compartments 2 may contain fragrance materials 25a, 25b, and 25c therein, as described with reference to FIG. 2A. By relatively rotating base 14 and first member 13, the amount of exposure of fragrance material 25d can range from substantially 100% in the position as shown in FIG. 4 to substantially 0% when first member 13 is rotated approximately 90° in either direction. During such rotation, the exposure of the appropriate adjacent fragrance material 25a or 25c will change from substantially 0% to substantially 100%. At rotations less than 90°, for this example, there will be a proportional exposure of the fragrance materials in adjacent quadrants with a resulting mixture, also called a blend, of the two fragrances emitted from the exposed compartments. First member 13 and base 14, as shown in FIG. 4, may be rotated relative to each other to result in blends of fragrances emitted from the fragrance materials in any two adjacent quadrants.

In other embodiments various sizes of the openings and number of the openings in the first member may be used. For example, in FIG. 7, two openings 42a and 42b are present in first member 44. In such a first member, using the quadrant example of FIG. 4, at least two fragrance materials will always be exposed. In addition, it is possible to have a blend of the fragrances from all four quadrants by selective rotation of the first member relative to the base.

In one embodiment, a compartment 2 may have a fragrance material 25 that is different than the fragrance material in at least one other compartment 2. In a base similar to that in FIG. 2A, for example, there may be two to twelve different fragrance materials. A first member for such a base may have an opening of approximately 1/12 of a circle, and operate to produce a blended fragrance as previously described. In another example configuration, first member 13 of FIG. 4 may have an opening extending over three compartments. If each compartment contains a different fragrance material, at least three fragrances are blended. In one embodiment, at least one of the volatile compositions located in a compartment comprises a different volatile composition than that in at least one other compartment.

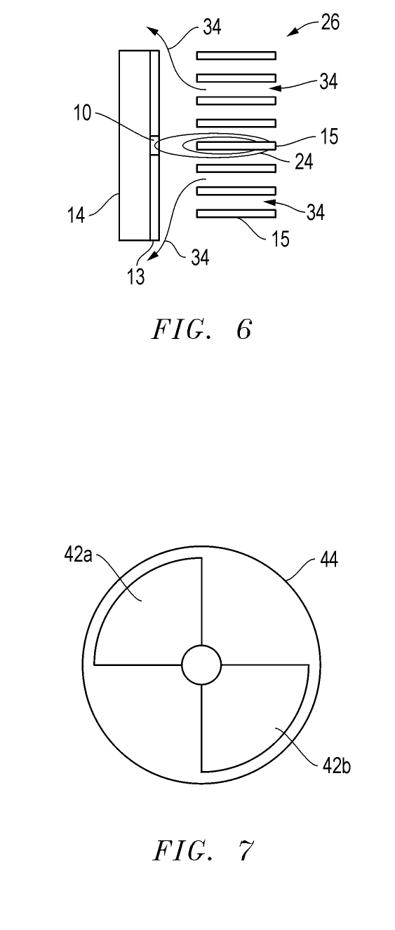

In one illustrative example, also referring to FIG. 6, opening 12 allows flowing ambient air from, for example, an air conditioner of a vehicle (not shown), to blow onto the fragrance material in the exposed compartments of base 14, thereby activating a release of fragrance from the fragrance material to the air inside the vehicle. The flowing ambient air includes cooled air, heated air, filtered air, humidified air, dehumidified air, and combinations thereof. FIG. 6 shows a side view of dispenser 1. Base 14, first member 13, and a mounting clip 24 are shown. In one embodiment, mounting clip 24 is attached to tab 10 of first member 13. Mounting clip 24 may be attached to any of ventilation louvers 15 of vent 26 of the vehicle. In this example, first member 13 does not rotate, but acts as an opening to the selected fragrance material. This side view shows the flow of air 34 from the vent 26 through the opening 12 in the first member 13 (not visible in FIG. 6) over the selected fragrance materials in base 14 and outward into the car interior. The fragrance from the fragrance material, or the blend of fragrances from the fragrance materials, exposed in the opening is dispersed into the surrounding atmosphere.

Referring to FIGS. 1 and 5, in one embodiment, second member 9 is a substantially circular disc rotatably mounted on center post 7 outside of first member 13. Second member 9 has an opening 62 and an adjuster tab 32 and may be rotated to further control the exposure of volatile composition, for example, fragrance material 25d, to ambient air of the surrounding atmosphere.

In operation, in one example, first member 13 may be rotated to expose selected compartments 2 containing fragrance materials 25. In addition, second member 9 may be rotated relative to first member 13 to restrict the flow of ambient air past the exposed compartments 2.

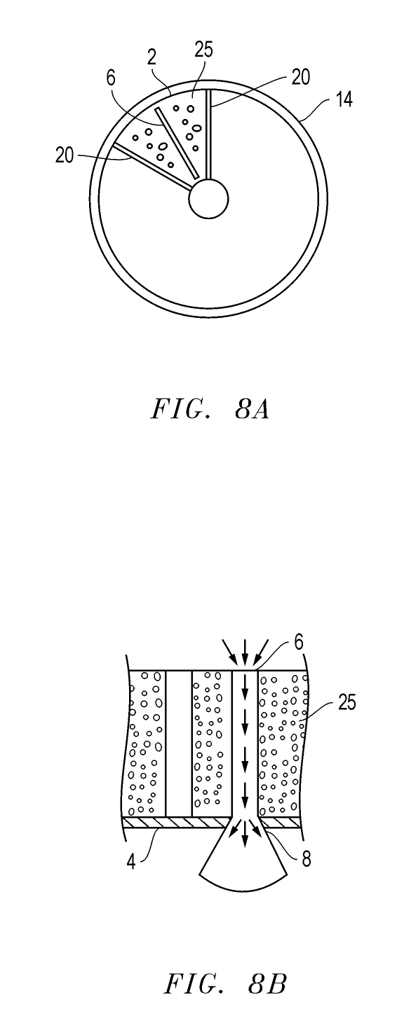

Referring to FIG. 8A, in one embodiment of the invention, a porous material having volatile composition 25 therein, for example a gel volatile composition, is in the form of a fragrance fin 6 extending above the level of the volatile composition 25 in compartment 2. The porous material may include an expanded plastic foam material. In another embodiment, the fragrance material itself may be formed into a solid fin. In another embodiment, fragrance fin 6 is used in each compartment without additional gel volatile composition in the compartment. A plurality of fins 6 may be present in each compartment 2. In one example, the surface area of fragrance fin 6 in compartment 2 may provide more exposed area for fragrance release than the area present with only a gel volatile composition disposed in the compartment.

Also referring to FIG. 8B, in one embodiment, suitable materials are chosen for dispenser 1 and fragrance material 25 that permit suitable light transmission resulting in a light pipe effect when dispenser 1 is illuminated. FIG. 8B shows an enlarged diagram of the light pipe effect 8 through fragrance fin 6 and bottom 4 of the base under suitable conditions, such as the appropriate selection of materials for fragrance fin 6 and base 14 and the appropriate strength of light through fragrance material 25 and base 14. In one example of the light pipe effect, light enters the top edge of a transparent material and due to substantially total internal reflection, substantially the same amount of light comes out the bottom. In order to obtain the light pipe effect with dispenser 1, fragrance material 25 and fragrance fins 6 would preferably be made of materials which permit the light to be at least partially transmitted through the apparatus. This permits the entire apparatus to glow and diffuse light and provide observable illumination. In order to achieve the desired illumination effect, the composition of the fragrance fins and the material of the apparatus, or at least the base, should be light transmitting. A light transmitting material has the ability to transmit light through the material so that the entire apparatus appears illuminated. Materials having suitable light transmission properties include, for example, clear polystyrene, clear polymethyl methacrylate, clear polycarbonate, polyethylene terephthalate and any other suitable clear plastic and/or thermoplastic.

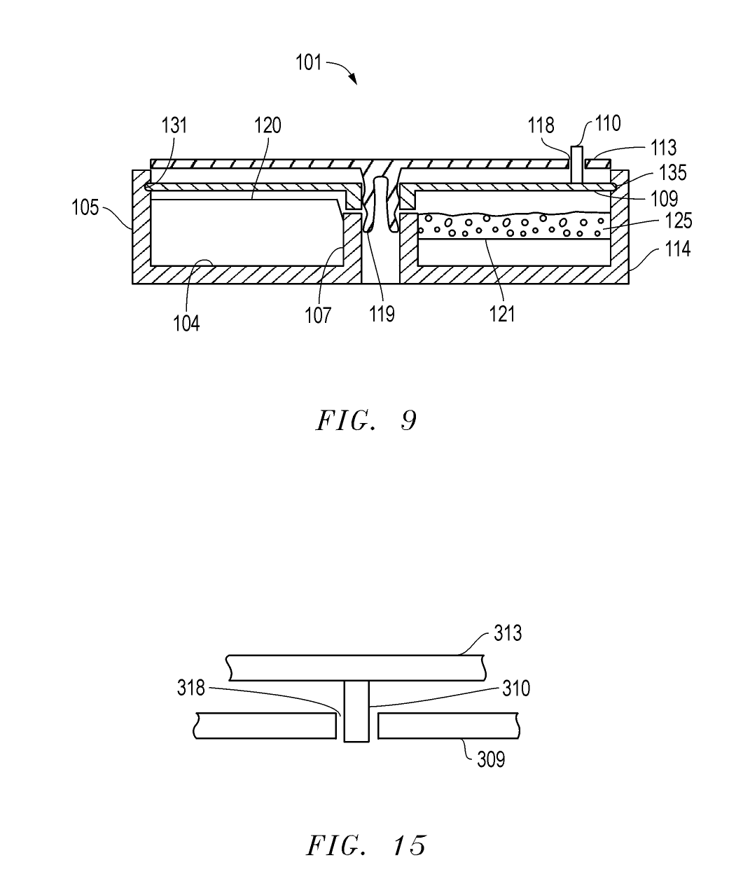

In another embodiment, referring to FIGS. 9-13, a dispenser 101 comprises base 114 having multiple compartments 102 (see FIG. 10A) separated by dividers 120. Base 114 has side wall 105 that has a circumferential groove 131 near a top end thereof. Center post 107 extends upward from bottom 104 of base 114 and maintains first member 109 and second member 113 in a substantially coaxial arrangement. Spring fingers 119 are attached to second member 113 and extend into center post 107 to retain second member 113 in the assembly. The friction exerted by spring fingers 119 is sufficient to hold second member 113 in place but still allows rotation of second member 113 relative to base 114 through, for example, the use of finger pressure by a user. For purposes of this example, the volatile composition will be discussed as an air freshening fragrance material. It is understood that any of the other volatile composition types discussed previously are also suitable for use in dispenser 101.

As shown more clearly in FIG. 10A, in this example, base 114 comprises three fragrance material zones L, M, and N corresponding to three compartments 102L-n containing fragrance materials 125L-n, respectively. Each compartment 102L-n has a short vane 121 approximately radially bisecting each compartment. Vane 121 facilitates anchoring fragrance material 125L-n in their respective compartments. FIG. 10B is a cross section as indicated in FIG. 10A that shows the relative heights of divider 120 and vane 121.

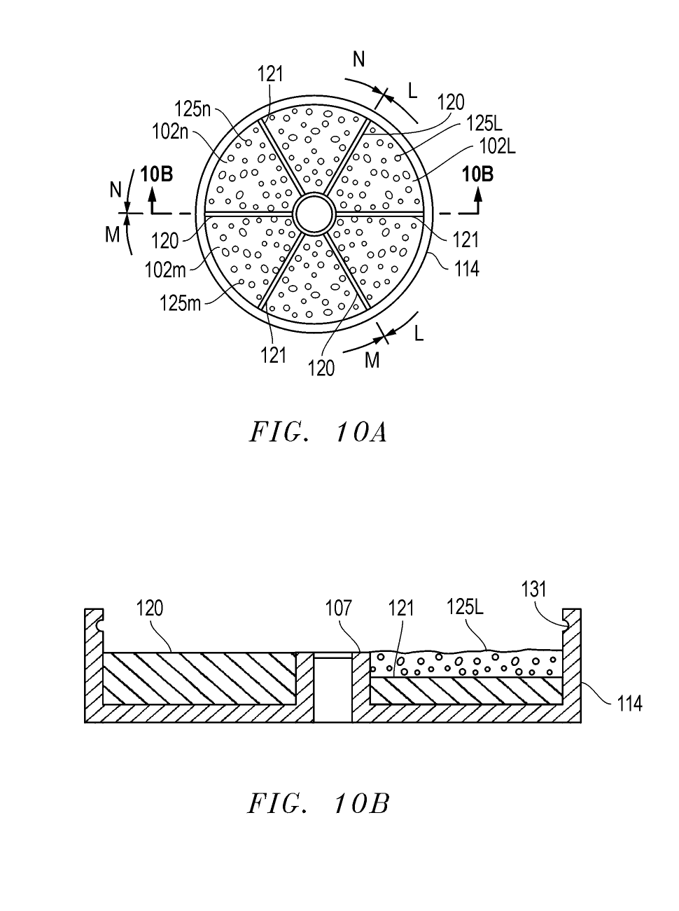

FIG. 11A shows a top side of second member 113 where second member 113 is substantially circular having multiple circumferential openings 117 extending angularly over an angle α of the second member 113 surface to allow air to flow through. Groove 118 extends over an angle θ on the opposite side of second member 113. In one embodiment, the angles α and θ are substantially equal and are also substantially equal to the angular dimension of a single compartment 102. For the example shown in FIGS. 9-13, the angle is about 120°. For different numbers of compartments, the angles α and θ may be different. In one embodiment, a stiffener 150 extends across grooves 117 and acts to stiffen the solid circumferential sections between grooves 117. Stiffener 150 may also provide a finger rest for facilitating rotation of second member 113 relative to base 114 (see FIG. 9). Multiple angularly spaced apart stiffeners may be used. Alternatively, instead of circumferential grooves 117, any other opening shape may be used, for example, radially extending grooves, a pattern of smaller holes extending over the second member 113 surface, or an opening substantially similar to opening 112 that is subtended by the angle α.

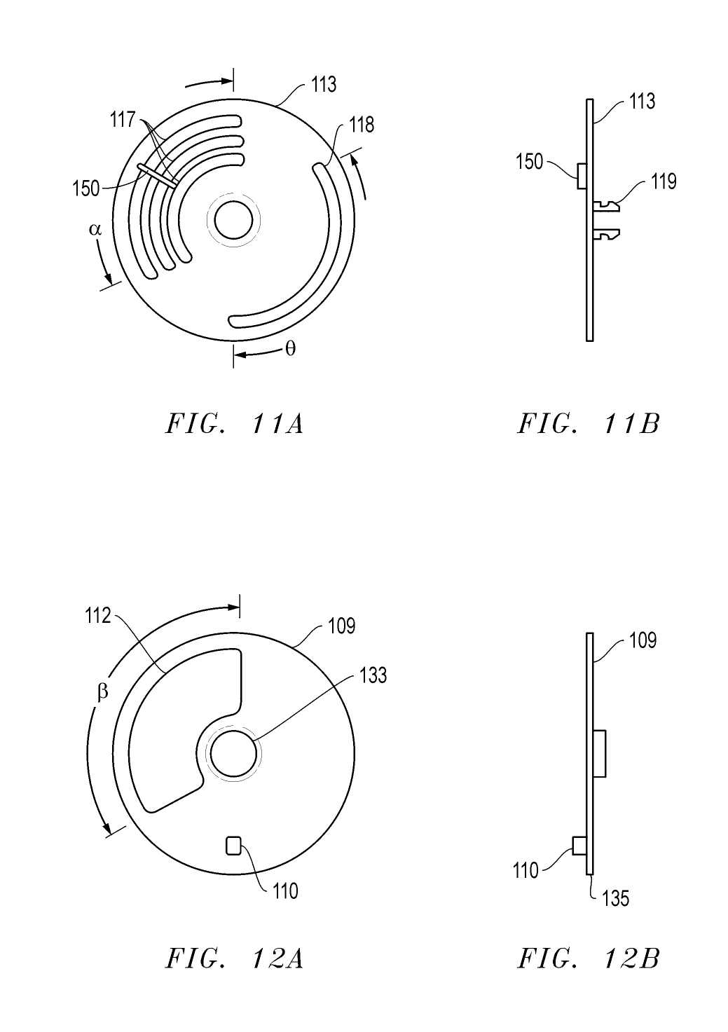

FIG. 11B is a side view of second member 113 showing spring fingers 119 suitable for rotatably attaching second member 113 to base 114. In one embodiment, spring fingers 119 extend through center hole 133 of first member 109 (see FIG. 12A), providing additional support and guidance to first member 109 in assembled dispenser 101. In one embodiment, circumferential edge 135 is sized to slidingly fit in groove 131 of base 114 such that first member 109 may be easily rotated in groove 131.

An example of a first member 109 is shown in FIGS. 12A and 12B. First member 109 has opening 112 extending over an angle β, as shown. In one embodiment, opening 112 covers about one third of the surface area of first member 109, such that angle β is approximately the same as angles α and θ. Tab 110 extends outward from the top surface of first member 109 and is located to fit in groove 118 of second member 113 at assembly. Tab 110 and groove 118 act cooperatively to define the range of rotation of first member 109 and second member 113 relative to each other.

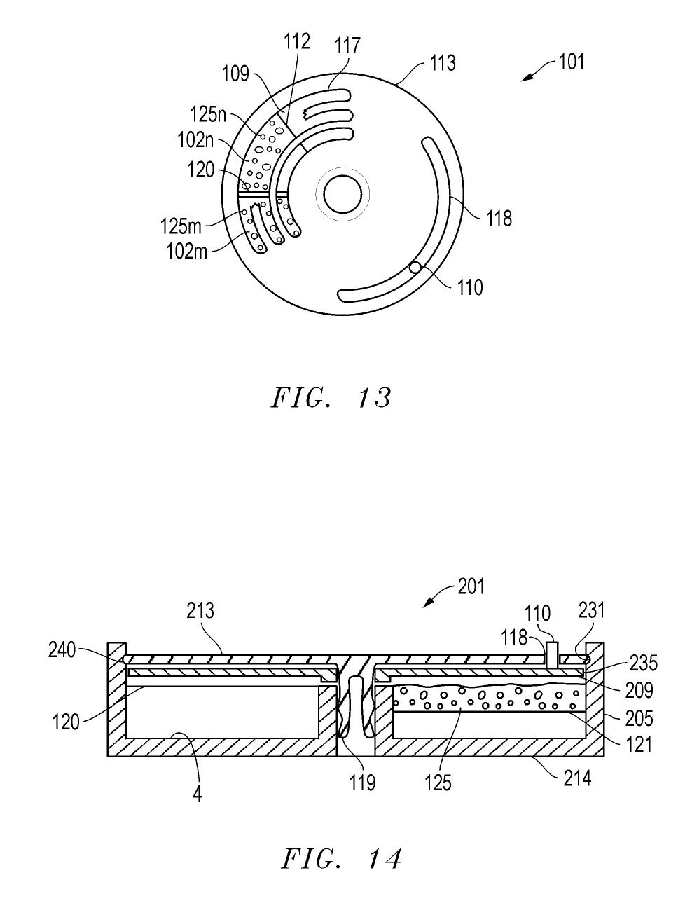

FIG. 13 shows a partial cutaway of one embodiment of dispenser 101 showing the interaction of various parts. Looking down on dispenser 101, second member 113 is partially cut away to show the position of first member 109 in relation to compartments 102m and 102n having fragrance materials 125m and 125n respectively therein. First member 109 has been rotated with respect to base 114 (not shown in this view) to expose portions of compartments 102m and 102n thereby exposing the respective fragrance materials in those compartments. This exposure allows the blended fragrance, from fragrance materials 125m and 125n, to disperse into the atmosphere surrounding dispenser 101. The position of first member 109, and opening 112, may be adjusted by moving tab 110 in groove 118. For example, a user may adjust the position of first member 109, and opening 112, by moving tab 110 in one direction or the opposite direction. The adjustment of the positions of second member 113 and first member 109 relative to base 114, and to each other, adjusts how much of the respective compartments are exposed, and therefore, which fragrances are blended. Such adjustments may be made, for example, by finger pressure on tab 110, second member 113, base 114, and combinations thereof.

When the desired position of first member 109 is beyond the limit provided by groove 118, both first member 109 and second member 113 may be rotated until first member 109 is in the desired position. Then, second member 113 may be rotated to adjust an overlap area defined by overlapping openings 112 and 117, as desired. Alternatively, second member 113 may be positioned to expose the desired fragrance material 102, and first member 109 may be rotated relative to second member 113 to adjust the overlap area by overlapping openings 112 and 117, as desired. It will be appreciated that iterative movement of both first member 109 and second member 113 may be required to achieve a desired fragrance, or fragrance blend, at a desired strength.

For example, assuming angles α, β, and θ are all substantially equal to a third of a circle, then first member 109 may be rotated to a position such that opening 112 exposes a single compartment 102, or a portion of two contiguous compartments 102. In addition, second member 113 may be rotated to adjust the amount of overlap area exposed by the combined openings created by the overlap of openings 112 and 117. Alternatively, still assuming angles α, β, and θ are all substantially equal to a third of a circle, second member 113 may be rotated to a position such that opening 117 exposes a single compartment 102, or a portion of two contiguous compartments 102. In addition, first member 109 may be rotated to adjust the amount of the overlap area by overlapping openings 112 and 117.

Tab 110 and groove 118 act cooperatively to define the range of rotation of first member 109 with respect to second member 113, which, therefore, defines the overlap area of overlapping openings 112 and 117. For example, when tab 110 is at one end of groove 118 (the lower end in FIG. 13), openings 112 and 117 are completely overlapped such that the entire area of opening 112, and the fragrance material subtended by opening 112, are exposed to the surrounding atmosphere. In contrast, when tab 110 is at an opposite end of groove 118 (the upper end in FIG. 13), there is no overlap of openings 112 and 117, and there is substantially no exposure of opening 112, and the fragrance material subtended by opening 112, to the surrounding atmosphere.

While described with reference to three compartments, the embodiments described in FIGS. 9-13 may be modified to have any suitable number of compartments. Likewise, the size of the opening 112 in first member 109 may be selected to subtend a larger or smaller arc than the circumferential grooves 117 in second member 113. Alternatively, the circumferential grooves 117 in second member 113 may be selected to subtend a larger or smaller arc than the opening 112 in first member 109.

In another embodiment, see FIG. 14, dispenser 201 has base 214 with first member 209 and second member 213. First member 209 is similar in form to first member 109. First member 209, however, is sized such that outer circumferential edge 235 of first member 209 is smaller than the inside diameter of side wall 205. Second member 213 is sized such that outer circumferential edge 240 of second member 213 may be captured by circumferential groove 231 in side wall 205. Other features indicated are similar to those described in FIGS. 9-13. Alternatively, base 214 may have two spaced apart circumferential grooves (not shown) for receiving first member 209 and second member 213.

While the first member and second member, and the interaction of the first member and the second member, have been described above in an illustrative geometry, it is intended that the present invention encompass other geometries. For example, in another embodiment, see FIG. 15, tab 310 may be located on the bottom side of second member 313 and fit into groove 318 in the first member 309.

Any of the dispensers described herein, including their individual parts, may be manufactured from any suitable material including, for example, plastic, thermoplastic, and metal. In one embodiment, at least portions of such dispensers are made from substantially transparent and/or translucent materials, for example, plastic and/or thermoplastic, to allow a user to view and adjust the relative positions of the movable members relative to the volatile composition compartments. In one embodiment, at least portions of such dispensers are made from polyethylene terephthalate. In one embodiment, coloring may be added to the volatile compositions to allow a user to select a desirable volatile composition or blend of volatile compositions.

The foregoing description is intended only as a means of illustration and should not be construed as limiting the scope of the invention in any way. Those skilled in the art will recognize many variations that may be made without departing from the spirit of the disclosed invention.

What is claimed is:

1. An apparatus comprising:
a base having a plurality of compartments;
a volatile composition located in at least one of the plurality of compartments; and
a first member in a substantially coaxial arrangement with the base, the first member having a first opening exposing at least a portion of at least one compartment to a surrounding atmosphere.

2. The apparatus of claim 1 further comprising a second member substantially coaxial with the first member, the second member having a second opening rotatably positionable relative to the first opening adjusting an overlap area.

3. The apparatus of claim 2 further comprising a tab attached to one of the first member and the second member, the tab acting cooperatively with a groove in the other of the first member and the second member to define a range of rotatable motion of the first member and the second member relative to each other.

4. The apparatus of claim 1 further comprising a circumferential groove in a wall of the base.

5. The apparatus of claim 4 wherein the first member is substantially circular and is sized to fit and rotate within the circumferential groove.

6. The apparatus of claim 1 wherein the volatile composition comprises a plurality of volatile compositions.

7. The apparatus of claim 6 wherein at least one of the volatile compositions located in one of the compartments comprises a different volatile composition than that in at least one other compartment.

8. The apparatus of claim 6 wherein at least one of the plurality of volatile compositions comprises a fragrance material.

9. The apparatus of claim 1 wherein the first member is rotatably positionable to expose at least a portion of at least two compartments to the surrounding atmosphere.

10. The apparatus of claim 9 wherein the at least two compartments comprise at least two contiguous compartments.

11. An apparatus comprising:
a base having a plurality of compartments wherein at least one of the plurality of compartments contains a volatile composition;
a first member in a substantially coaxial arrangement with the base, the first member having a first opening exposing a portion of two contiguous compartments to a surrounding atmosphere; and
a second member substantially coaxial with the first member having a second opening rotatably positionable relative to the first opening adjusting an overlap area.

12. The apparatus of claim 11 further comprising a tab attached to one of the first member and the second member, the tab acting cooperatively with a groove in the other of the first member and the second member to define a range of rotatable motion of the first member and the second member relative to each other.

13. The apparatus of claim 11 further comprising a circumferential groove in a wall of the base and wherein one of the first member and the second member is substantially circular and is sized to fit and rotate within the circumferential groove.

14. The apparatus of claim 11 wherein the volatile composition comprises a fragrance material.

15. A method comprising:
placing a volatile composition in at least one of a plurality of compartments of a base; and
rotating a first member in a substantially coaxial arrangement with the base such that a first opening in the first member exposes at least a portion of at least one compartment to a surrounding atmosphere.

16. The method of claim 15 further comprising rotating a second member substantially coaxial with the first member such that a second opening in the second member acts cooperatively with the first opening adjusting an overlap area.

17. The method of claim 15 further comprising defining a range of rotational motion of the first member and the second member relative to each other.

18. The method of claim 17 wherein defining the range of rotational motion of the first member and the second member relative to each other comprises positioning a tab attached to one of the first member and the second member into a groove in the other of the first member and the second member.

19. The method of claim 15 wherein rotating the first member comprises rotating the first member rotatably coupled to the base such that the first opening in the first member exposes at least two compartments to the surrounding atmosphere.

20. The method of claim 15 wherein the volatile composition comprises a plurality of volatile compositions.
